(19) United States
(12) Patent Application Publication
(10) Pub. No.: US 2023/0122455 A1
(43) Pub. Date: Apr. 20, 2023

(54) PROGNOSTIC MARKERS OF DISEASE RECURRENCE IN LIVER TRANSPLANT RECIPIENTS WITH HEPATOCELLULAR CARCINOMA

(71) Applicant: Universiteit Gent, Gent (BE)

(72) Inventors: Xavier Verhelst, Landskouter (BE); Hans Van Vlierberghe, Kruisem (BE)

(21) Appl. No.: 17/907,395

(22) PCT Filed: Mar. 25, 2021
(86) PCT No.: PCT/EP2021/057788

§ 371 Date: Sep. 26, 2022

(30) Foreign Application Priority Data
Mar. 27, 2020 (EP) 20166316.8

(51) Int. Cl. G01N 33/574

(52) U.S. Cl. CPC G01N 33/57438; G01N 2400/12; G01N 2800/52

(57) ABSTRACT

Predicting recurrence of hepatocellular carcinoma (HCC) after liver transplantation. The disclosure indeed discloses a set of biomarkers present in a serum sample taken from a HCC patient before liver transplantation, which can be used to assess recurrence of HCC after liver transplantation. More specifically, the disclosure discloses a process to predict the recurrence of HCC after liver transplantation via determining the amount of at least four specific N-glycans in a serum sample.

### CROSS-REFERENCE TO RELATED APPLICATIONS

This application is a national phase entry under 35 U.S.C. § 371 of International Patent Application PCT/EP2021/057788, filed Mar. 25, 2021, designating the United States of America and published as International Patent Publication WO 2021/191372 Al on Sep. 30, 2021, which claims the benefit under Article 8 of the Patent Cooperation Treaty to European Patent Application Serial No. 20166316.8, filed Mar. 27, 2020.

### TECHNICAL FIELD

The disclosure relates to the technical field of predicting recurrence of hepatocellular carcinoma (HCC) after liver transplantation. The disclosure indeed discloses a set of biomarkers present in a serum sample taken from a HCC patient before liver transplantation, which can be used to assess recurrence of HCC after liver transplantation. More specifically, the disclosure discloses a process to predict the recurrence of HCC after liver transplantation via determining the amount of at least four specific N-glycans in a serum sample.

### BACKGROUND

Liver transplantation is the ultimate treatment for end stage liver disease and selected patients with hepatocellular carcinoma. Only patients with HCC limited to the liver (without extrahepatic disease) and responding to strict criteria regarding the number and size of the HCC lesions (e.g., Milan-Criteria, Up-to-seven Criteria, . . . ) can be considered for liver transplantation in order to limit the risk of disease recurrence after transplantation. However, although these criteria show a strong correlation with disease recurrence, sensitivity and specificity should be increased, as recurrence still occurs despite stringent application of these criteria. This could be explained by underestimation of the number of lesions using current imaging modalities and because tumor biology is not taken into account in these models. The recent Duvoux-model (Duvoux et al. 2012) takes into account alpha-fetoprotein, a diagnostic marker for HCC, to increase the predictive power of HCC recurrence. On the other hand, some patients are denied liver transplantation based on these models, although their risk of recurrence of HCC after liver transplantation could be low.

There is thus a clear need for prognostic biomarkers that assess the risk of HCC recurrence after liver transplantation, beyond the current models.

Miyahara et al. (2014 & 2015) and Kamiyama et al. (2013) disclose that specific serum glycans in patients with HCC present before treatment is initiated, are independently associated with HCC recurrence and overall survival. However, it has not been studied and is unknown whether and which set of serum glycans can be efficiently used to predict with high certainty the recurrence of HCC after liver transplantation.

### BRIEF DESCRIPTION OF THE DRAWINGS

FIG. 1. Cumulative risk of HCC recurrence after LT according to HCC Recurrence Score LR. Log Rank test: p=0.017.

FIG. 2. Cumulative risk of HCC recurrence after LT according to HCC Recurrence Score CR. Log Rank test: p=0.017.

### DETAILED DESCRIPTION

The disclosure relates in first instance to a process to predict recurrence of hepatocellular carcinoma after liver transplantation comprising:
providing a serum or plasma sample obtained from a hepatocellular carcinoma patient before the patient receives a liver transplant,
determining the amount of the N-glycans: agalacto, core-alpha-1,6-fucosylated biantennary glycan (NGA2F), an agalacto, core-alpha-1,6-fucosylated bisecting biantennary (NGA2FB), an unfucosylated triantennary glycan (NA3), and a branching alpha-1,3-fucosylated and core-alpha-1,6-fucosylated triantennary glycan (NA3Fbc) within the serum or plasma sample,
wherein decreased amounts of NGA2F and NGA2FB, and, increased amounts of NA3 and NA3Fbc—compared to the amounts of NGA2F, NGA2FB, NA3 and NA3Fbc within a control serum sample—are predictive for recurrence of hepatocellular carcinoma after liver transplantation.

The terms ‘to predict recurrence of hepatocellular carcinoma after liver transplantation’ relates to forecasting in a reliable manner whether a recipient—who has/had HCC before transplantation—will develop HCC after transplantation.

The terms ‘providing a serum or plasma sample obtained from a HCC patient’ relate to well-known practices to obtain a blood sample—and subsequently a serum or plasma sample—from a human being. The disclosure thus relates to an ‘in vitro’ diagnostic method applied on a serum or plasma sample after the serum/plasma sample has been removed from a human body.

The term agalacto, core-alpha-1,6-fucosylated biantennary glycan (NGA2F) refers to the following oligosaccharide-type structure:

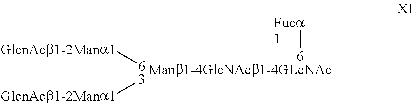

The term agalacto, core-alpha-1,6-fucosylated bisecting biantennary glycan (NGA2FB) relates to the following oligosaccharide-type structure:

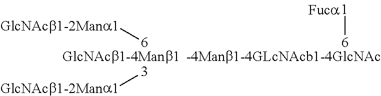

The term unfucosylated triantennary glycan (NA3) relates to the following oligosaccharide-type structure:

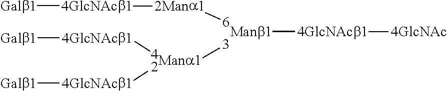

The term branching alpha-1,3 fucosylated and core-alpha-1,6-fucosylated triantennary glycan (NA3Fbc) relates to the following oligosaccharide-type structure :

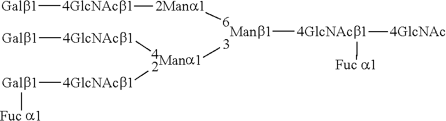

The disclosure further relates to a process as describe above wherein, in addition, the amount of core-alpha-1,6-fucosylated triantennary glycan (NA3Fc) is determined within the serum/plasma sample and wherein an increased amounts of NA3Fc—compared to the amounts of NA3Fc within a control serum/plasma sample—is predictive for recurrence of hepatocellular carcinoma after liver transplantation.

The term core-alpha-1,6-fucosylated triantennary glycan (NA3Fc) relates to the following oligosaccharide-type structure:

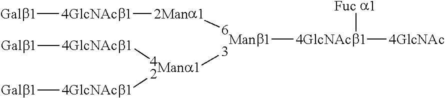

The terms “determining the amount of the N-glycans: agalacto, core-alpha-1,6-fucosylated biantennary (NGA2F), agalacto, core-alpha-1,6-fucosylated bisecting biantennary (NGA2FB), unfucosylated triantennary glycan (NA3), branching alpha-1,3-fucosylated and core alpha-1,6-fucosylated triantennary glycan (NA3Fbc) and core-alpha-1,6-fucosylated triantennary glycan (NA3Fc) within the serum sample” ‘relates to any method known to (relatively) quantify the presence of NGA2F, NGA2FB, NA3, NA3Fbc and/or NA3Fc molecules within the serum sample. Specifically, the latter terms refer to ‘determining the amount of NGA2F, NGA2FB, NA3, NA3Fbc and/or NA3Fc via capillary electrophoresis’ and more specifically the latter terms refer to ‘determining the amount of NGA2F, NGA2FB, NA3, NA3Fbc and/or NA3Fc via DNA sequencer-assisted fluorophore-assisted capillary electrophoresis’ as is described by Callewaert et al. (2004 Nat Med 10: 429). Other techniques can be used for quantification/profiling of glycans including MALDI-TOF Mass spectometry, high-performance anion-exchange chromatography with pulsed amperometric detection, High Performance Liquid chromatography and Lectin Array.

The preparation and labeling of the glycans in serum sample for sequencing can be performed using the on-membrane deglycosylation method described by Laroy et al. (Nat. Protoc. 1, 397-405 (2006)). An in-solution deglycosylation method has been developed (Laroy et al. 2006) for serum glycan profiling in a clinical context (sample preparation time less than 2 hours).

The disclosure thus specifically relates to a process as described above wherein the determining the amount of the glycans within the serum/plasma sample is undertaken by capillary electrophoresis. Moreover, the disclosure relates to a process as described above wherein the capillary electrophoresis is DNA sequencer-assisted fluorophore-assisted capillary electrophoresis.

The disclosure further relates to a process as described above wherein the control serum/plasma sample is a serum/plasma sample obtained from a hepatocellular carcinoma patient before the patient receives a liver transplant and wherein the patient experiences no recurrence of hepatocellular carcinoma after liver transplantation.

The disclosure thus relates to the usage of the N-glycans NGA2F, NGA2FB, NA3, NA3Fbc and/or NA3Fc to predict recurrence of hepatocellular carcinoma after liver transplantation according to a process as described above. Based on changes in relative abundance of glycans in patients who show recurrent HCC after liver transplantation, the following scoring systems can, for example, be used: a “HCC Recurrence Score LR,” which calculates a risk score using a logistic regression model, or, a “HCC Recurrence Score CR,” which calculates a risk score using a cox regression model.

Based on the latter Recurrence Scores a ROC curve analysis can be undertaken and—based on, for example, the Youden-index—a cut-off with the best matching sensitivity and specificity can be calculated.

The disclosure will now be further illustrated by the following non-limiting examples.

Examples

In a prospective monocentric study in an expert liver transplant unit, consecutive serum samples were collected the day before liver transplantation (LT) in 255 patients between 2 Jul. 2011 and 24 Sep. 2018. In this cohort, 76 patients were diagnosed having HCC. Patients were followed after LT for at least one year. Serum glycomic analysis was performed as described by Callewaert et al. and Laroy et al. using capillary electrophoresis. This yields an electropherogram with 13 peaks covering the entire spectrum from agalactosylated biantennary glycans to complex tetra-antennary glycans. The relative abundance of these glycans is normalized over the total amount of measured glycans in the serum sample.

In this cohort, 8 patients developed HCC recurrence during follow up. The pre-transplant glycomic profile of these patients was significantly different of patients with HCC who did not develop HCC recurrence after liver transplantation.

Using both logistic and cox regression, a specific glycomic fingerprint was observed as described before, and summarized in table 1 and 2.

TABLE 1
Univariate Logistic Regression analysis of changes in
abundance of serum glycans in patients with recurrent
HCC after liver transplantation compared to HCC patients
without recurrence after liver transplantation.

| | Regressie | | | | |
|---|---|---|---|---|---|
| | coefficient | OR | p value | 95% CI | 95% CI |
| NGA2F | −0.036 | 0.965 | 0.011 | 0.965 | 0.992 |
| NGA2FB | −0.17 | 0.863 | 0.009 | 0.772 | 0.964 |
| NG1A2F | −0.04 | 0.960 | 0.073 | 0.918 | 1.004 |
| NG1A2Fisomer | 0.013 | 1.013 | 0.655 | 0.957 | 1.073 |
| NA2 | 0.004 | 1.004 | 0.377 | 0.996 | 1.012 |
| NA2F | 0.002 | 1.003 | 0.803 | 0.983 | 1.023 |
| NA2FB | −0.024 | 0.976 | 0.071 | 0.951 | 1.002 |
| NA3 | 0.033 | 1.034 | 0.010 | 1.008 | 1.060 |
| NA3Fb | 0.027 | 1.028 | 0.259 | 0.980 | 1.079 |
| NA3Fc | 0.085 | 1.202 | 0.003 | 1.018 | 1.419 |
| NA3Fbc | 0.071 | 1.075 | 0.031 | 1.006 | 1.147 |
| NA4 | 0.050 | 1.054 | 0.612 | 0.860 | 1.293 |
| NA4Fb | −0.072 | 0.930 | 0.675 | 0.663 | 1.304 |

TABLE 2
Univariate Cox Regression analysis of changes in abundance
of serum glycans in patients with recurrent HCC after
liver transplantation compared to HCC patients without
recurrence after liver transplantation.

| | Regressie | | | | |
|---|---|---|---|---|---|
| | coefficient | HR | p value | 95% CI | 95% CI |
| NGA2F | −0.029 | 0.972 | 0.018 | 0.949 | 0.995 |
| NGA2FB | −0.110 | 0.896 | 0.013 | 0.822 | 0.977 |
| NG1A2F | −0.033 | 0.967 | 0.106 | 0.929 | 1.007 |
| NG1A2Fisomer | 0.011 | 1.011 | 0.684 | 0.959 | 1.066 |
| NA2 | 0.002 | 1.002 | 0.538 | 0.995 | 1.010 |
| NA2F | 0.001 | 1.001 | 0.873 | 0.984 | 1.019 |
| NA2FB | −0.018 | 0.982 | 0.107 | 0.960 | 1.004 |
| NA3 | 0.020 | 1.020 | 0.001 | 1.008 | 1.032 |
| NA3Fb | 0.022 | 1.022 | 0.303 | 0.980 | 1.066 |
| NA3Fc | 0.004 | 0.996 | 0.732 | 0.974 | 1.018 |
| NA3Fbc | 0.034 | 1.035 | 0.009 | 1.009 | 10.62 |
| NA4 | 0.036 | 1.036 | 0.703 | 0.863 | 1.245 |
| NA4Fb | −0.028 | 0.972 | 0.847 | 0.732 | 1.291 |

Calculation of the HCC Recurrence Score Log Reg and the HCC Recurrence Score Cox Reg:

Based on this information first an HCC recurrence Score based on logistic regression was calculated as:

HCC Recurrence Score Log Reg=(NGA2F*(−0.036))+(NGA2FB*(−0.147))+(NA3*0.033)+(NA3Fc*0.182)+(NA3Fbc*0.071)

A similar score was developed using Cox regression analysis:

HCC Recurrence Score Cox Reg=(NGA2F*(−0.029))+(NGA2FB*(−0.110))+(NA3*0.020)+(NA3Fc*0.031)+(NA3Fbc*0.034)

ROC curve analysis showed an AUC of 0.866 (p=0.001; 95% CI 0.750-0.982) for the HCC Recurrence Score Log Reg and an AUC of 0.855 (p=0.001; 95% CI 0.731-0.979) for the HCC Recurrence Score Cox Reg for the development of recurrent HCC after liver transplantation.

Based on the Youden index, a cut-off of −3.2788 was defined for the HCC Recurrence Score Log Reg showing a sensitivity of 87.5% and a specificity of 73.5%.

For the HCC Recurrence Score Cox Reg, a cut-off of −4.24 was defined using a similar approach. Sensitivity was 87.5% and specificity 67.6%.

Using this cut-off, the Kaplan Meier curve showed a significant difference for the HCC Recurrence Score Log Reg score (Log Rank test: p=0.001) between patients with and without HCC recurrence. The cumulative incidence of HCC recurrence was 2% in patients below this cutoff and 28% in patients with a pre-transplant value above this cutoff.

When the HCC Recurrence Score Cox Reg was applied, the cumulative incidence of HCC was 2.1% after liver transplantation if the value was below −4.24, and 24.1% if the value was equal to or higher than this cut-off (Log Rank t test: p=0.011).

In a next step, the association of these scoring systems with HCC recurrence without using NA3Fc was calculated. This shows the independent and strong association of this glycomic signature with HCC recurrence after liver transplantation without the use of NA3Fc.

HCC Recurrence Score LR=(NGA2F*(−0.036))+(NGA2FB*(−0.17))+(NA3*0.033)+(NA3Fbc*0.071)

ROC curve analysis for the “HCC recurrence score LR” showed an area under the curve (AUC) of 0.837 (p=0.002; 95% CI 0.711-0.963). The Youden-index was used to calculate a cut-off with the best matching sensitivity and specificity. This was defined at −5.80 associated with a sensitivity of 87.5% and specificity of 68.1% for HCC recurrence after LT.

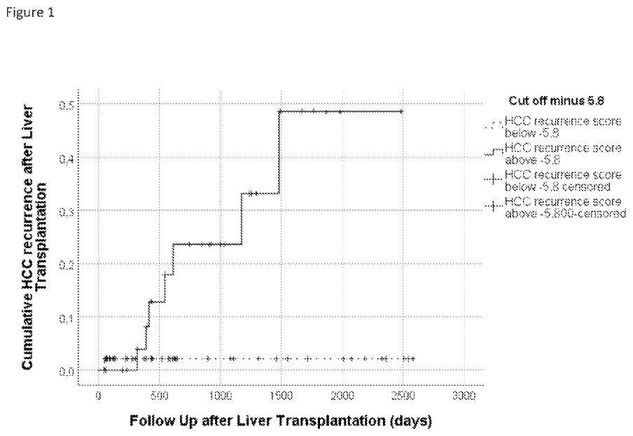

Dividing the cohort according to this cut-off shows a clear differentiation according to HCC recurrence risk (FIG. 1).

HCC Recurrence Score CR=(NGA2F*(−0.029))+(NGA2FB*(−0.110))+(NA3*0.020)+(NA3Fbc*0.034)

ROC curve analysis for the “HCC recurrence score CR” showed an area under the curve (AUC) of 0.837 (p=0.002; 95% CI 0.709-0.965). The Youden-index was used to calculate a cut-off with the best matching sensitivity and specificity. This was defined at −4.54 associated with a sensitivity of 87.5% and specificity of 68.1% for HCC recurrence after LT.

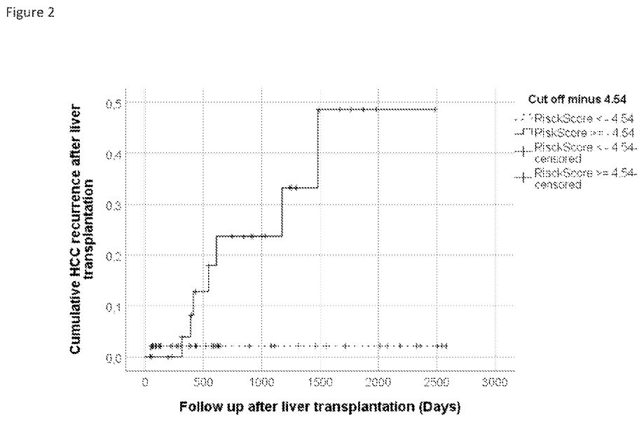

Dividing the cohort according to this cut-off shows a clear differentiation according to HCC recurrence risk (FIG. 2).

### REFERENCES

Duvoux C, Roudot-Thoraval F, Decaens T, Pessione F, Badran H, Piardi T, Francoz C, Compagnon P, Vanlemmens C, Dumortier J, Dharancy S, Gugenheim J, Bernard P H, Adam R, Radenne S, Muscari F, Conti F, Hardwigsen J, Pageaux G P, Chazouillères O, Salame E, Hilleret M N, Lebray P, Abergel A, Debette-Gratien M, Kluger M D, Mallat A, Azoulay D, Cherqui D; Liver Transplantation French Study Group. Liver transplantation for hepatocellular carcinoma: a model including α-fetoprotein improves the performance of Milan criteria. Gastroenterology. 2012 October ;143(4): 986-94.

Miyahara et al. Alteration of N-glycan profiles in patients with chronic hepatitis and hepatocellular carcinoma. Hepatology Research 2015: 986.

Miyahara et al. Serum glycan as a prognostic marker in patients with advanced hepatocellular carcinoma treated with sorafenib. Hepatology 2014: 355.

Kamayama et al. Identification of novel serum biomarkers of hepatocellular carcinoma using glycomic analysis. Hepatology 2013: 2314.

Callewaert, N. et al. Noninvasive diagnosis of liver cirrhosis using DNA sequencer-based total serum protein glycomics. Nat. Med. 10, 429-34 (2004).

Laroy, W., Contreras, R. & Callewaert, N. Glycome mapping on DNA sequencing equipment. Nat. Protoc. 1, 397-405 (2006).

1. A process to predict recurrence of hepatocellular carcinoma after liver transplantation, the process comprising:
providing a serum or a plasma sample obtained from a hepatocellular carcinoma patient before the patient receives a liver transplant, and
determining amounts of N-glycans: agalacto, core-alpha-1,6-fucosylated biantennary (NGA2F), agalacto, core-alpha-1,6-fucosylated bisecting biantennary (NGA2FB), triantennary glycan (NA3), and branching alpha-1,3-fucosylated and core-alpha-1,6-fucosylated triantennary glycan (NA3Fbc) within the serum or plasma sample,
wherein decreased amounts of NGA2F and NGA2FB, and, increased amounts of NA3 and NA3Fbc—compared to the amounts of NGA2F, NGA2FB, NA3 and NA3Fbc within a control serum or plasma sample—are predictive for recurrence of hepatocellular carcinoma after liver transplantation.

2. A process according to claim 1 wherein, in addition, the amount of core-alpha-1,6-fucosylated triantennary glycan (NA3Fc) is determined within the serum or plasma sample and wherein an increased amounts of NA3Fc—compared to the amounts of NA3Fc within a control serum or plasma sample—is predictive for recurrence of hepatocellular carcinoma after liver transplantation.

3. A process according to claim 1, wherein the determining the amount of the glycans within the serum or plasma sample is undertaken by capillary electrophoresis.

4. A process according to claim 3, wherein the capillary electrophoresis is DNA sequencer-assisted fluorophore-assisted capillary electrophoresis.

5. A process according to claim 1, wherein the patient experiences no recurrence of hepatocellular carcinoma after liver transplantation.

6. The process according to claim 2, wherein the determining the amount of the glycans within the serum or plasma sample is undertaken by capillary electrophoresis.

7. The process according to claim 6, wherein the capillary electrophoresis is DNA sequencer-assisted fluorophore-assisted capillary electrophoresis.

8. The process according to claim 2, wherein the patient experiences no recurrence of hepatocellular carcinoma after liver transplantation.

9. The process according to claim 3, wherein the patient experiences no recurrence of hepatocellular carcinoma after liver transplantation.

10. The process according to claim 4, wherein the patient experiences no recurrence of hepatocellular carcinoma after liver transplantation.

11. The process according to claim 6, wherein the patient experiences no recurrence of hepatocellular carcinoma after liver transplantation.

12. The process according to claim 7, wherein the patient experiences no recurrence of hepatocellular carcinoma after liver transplantation.

13. A process of performing a liver transplant in a patient having hepatocellular carcinoma, wherein the improvement comprises:
determining—in a serum or plasma sample obtained from the patient before the liver transplant—amounts of N-glycans: agalacto core-alpha-1,6-fucosylated biantennary (NGA2F), agalacto core-alpha-1,6-fucosylated bisecting biantennary (NGA2FB), triantennary glycan (NA3), and branching alpha-1,3-fucosylated and core-alpha-1,6-fucosylated triantennary glycan (NA3Fbc) within the serum or plasma sample, and
afterwards performing the liver transplant in the patient.

14. The process of claim 13, wherein decreased amounts of NGA2F and NGA2FB and increased amounts of NA3 and NA3Fbc—in comparison to amounts of NGA2F, NGA2FB, NA3 and NA3Fbc within a control serum or plasma sample—are predictive for recurrence of hepatocellular carcinoma after liver transplantation.

15. The process of claim 13, further comprising:
determining the amount of core-alpha-1,6-fucosylated triantennary glycan (NA3Fc) within the serum or plasma sample,
wherein an increased amount of NA3Fc—compared to the amounts of NA3Fc within a control serum or plasma sample—is predictive for recurrence of hepatocellular carcinoma after liver transplantation.

16. The process of claim 13, wherein determining the amount of the glycans within the serum or plasma sample is by capillary electrophoresis.

17. The process of claim 16, wherein the capillary electrophoresis is DNA sequencer-assisted fluorophore-assisted capillary electrophoresis.

18. The process of claim 13, wherein the patient experiences no recurrence of hepatocellular carcinoma after liver transplantation.

19. The process of claim 15, wherein the patient experiences no recurrence of hepatocellular carcinoma after liver transplantation.

20. The process of claim 16, wherein the patient experiences no recurrence of hepatocellular carcinoma after liver transplantation.
